(19) United States
(12) Patent Application Publication
(10) Pub. No.: US 2009/0187215 A1
(43) Pub. Date: Jul. 23, 2009

(54) METHODS AND APPARATUS TO REDUCE A DIMENSION OF AN IMPLANTABLE DEVICE IN A SMALLER STATE

(75) Inventors: David Mackiewicz, Scotts Valley, CA (US); Boris Anukhin, San Jose, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: Abbott Laboratories, Abbott Park, IL (US)

(21) Appl. No.: 12/338,977

(22) Filed: Dec. 18, 2008

Related U.S. Application Data
(60) Provisional application No. 61/015,144, filed on Dec. 19, 2007.

(51) Int. Cl. A61B 17/03; B29C 47/12; C23F 1/02; A61F 2/06

(52) U.S. Cl. 606/213; 264/177.17; 216/41; 623/1.15

(57) ABSTRACT

Devices and methods fabricating an implantable device are disclosed. A method of fabricating an implantable device is disclosed. The method includes positioning a planar base material. The planar base material has a first inner surface. The first inner surface has a first inner surface dimension. The planar base material has a first outer surface. The first outer surface has a first outer surface dimension. A portion of the first inner surface of the base material is removed to define an annular body movable from a first state towards a second state. The annular body includes a second inner surface having a second inner surface dimension and a second outer surface having a second outer surface dimension. The second inner surface dimension is smaller than the first inner surface dimension, the first outer surface dimension, and the second outer surface dimension.

### CROSS-REFERENCE TO RELATED APPLICATIONS

This application claims the benefit of and priority to U.S. Provisional Patent Application No. 61/015,144, filed Dec. 19, 2007, and entitled “Methods And Apparatus To Reduce A Dimension Of An Implantable Device In A Smaller State” which is incorporated herein by reference in its entirety. This application also incorporates U.S. patent application Ser. No. 11/767,818, entitled “Methods, Devices, and Apparatus for Managing Access Through Tissue”, and filed Jun. 25, 2007 by reference in its entirety.

### FIELD OF THE INVENTION

The present invention relates generally to medical devices, and more particular to methods and apparatuses to reduce a dimension of an implantable device in a smaller state.

### BACKGROUND OF THE INVENTION

Catheterization and interventional procedures, such as angioplasty or stenting, generally are performed by inserting a hollow needle through a patient's skin and tissue into the vascular system. A guide wire may be advanced through the needle and into the patients blood vessel accessed by the needle. The needle is then removed, enabling an introducer sheath to be advanced over the guide wire into the vessel, e.g., in conjunction with or subsequent to a dilator. Because these procedures are generally performed in arteries and other vasculature, it may be desirable to reduce the size of any components involved.

A catheter or other device may then be advanced through a lumen of the introducer sheath and over the guide wire into a position for performing a medical procedure. Thus, the introducer sheath may facilitate introducing various devices into the vessel, while minimizing trauma to the vessel wall and/or minimizing blood loss during a procedure.

Upon completing the procedure, the devices and introducer sheath would be removed, leaving a puncture site in the vessel wall. Traditionally, external pressure would be applied to the puncture site until clotting and wound sealing occur; however, the patient must remain bedridden for a substantial period of time after clotting to ensure closure of the wound. This procedure, however, may be time consuming and expensive, requiring as much as an hour of a physician's or nurse's time. It is also uncomfortable for the patient, and requires that the patient remain immobilized in the operating room, catheter lab, or holding area. In addition, a risk of hematoma exists from bleeding before hemostasis occurs.

Various apparatus have been suggested for percutaneously sealing a vascular puncture by occluding the puncture site. For example, U.S. Pat. Nos. 5,192,302 and 5,222,974, issued to Kensey et al., describe the use of a biodegradable plug that may be delivered through an introducer sheath into a puncture site. Another technique has been suggested that involves percutaneously suturing the puncture site, such as that disclosed in U.S. Pat. No. 5,304,184, issued to Hathaway et al.

Accordingly, it may be desirable to provide apparatus and methods to reduce a dimension of an implantable device in a smaller state.

### BRIEF SUMMARY

An embodiment of a method of fabricating an implantable device is described. The method includes positioning a planar base material having a first inner surface having a first inner surface dimension and a first outer surface having a first outer surface dimension. A portion of the first inner surface of the base material is removed to define an annular body movable from a first state towards a second state. The annular body includes a second inner surface having a second inner surface dimension and a second outer surface having a second outer surface dimension. The second inner surface dimension is smaller than the first inner surface dimension, the first outer surface dimension, and the second outer surface dimension.

Another embodiment of a method of fabricating an implantable device is described. The method includes extruding a base material having an inner surface and an outer surface to form a cross-section. The cross section is selected from the following: an isosceles trapezoid, a semipolygon, a triangle, or a semiellipse. The outer surface defines a base of the cross-section. An implantable device is formed from the extruded base material. The implantable device includes an annular body movable from a first state towards a second state. The implantable device is deformed from the first state where the implantable device has a first dimension to the second state where the implantable device has a second dimension. The first dimension in the first state is substantially larger than the second dimension in the second state.

In some embodiments, the second inner surface and the second outer surface define a cross-section. The shape of the cross-section, in further embodiments, is selected from the following: an isosceles trapezoid, a semipolygon, a triangle, or a semiellipse. The second outer surface, in some configurations, defines a base of the cross-section.

Removing a portion of the base material, in some embodiments, includes removing one or more portions from the base material using laser cutting. In further embodiments, removing a portion of the base material includes using a laser at an angle that is not perpendicular to the first outer surface or second outer surface.

In some embodiments, removing a portion of the base material includes removing a portion from the base material using photochemical etching. Removing a portion of the base material, in further embodiments, includes selectively adding an outer mask having an outer mask dimension to the first outer surface of the base material and selectively adding an inner mask having an inner mask dimension to the first inner surface of the base material to form the cross-section. In some embodiments, the outer mask dimension is larger than the inner mask dimension.

The first inner surface dimension and the first outer surface dimension, in some embodiments, are substantially the same dimension. In further embodiments, the method includes removing a portion of the first outer surface of the base material and the first outer surface dimension is substantially larger than the second outer surface dimension.

In some embodiments, the annular body includes a plurality of adjacent support members. The method, in further embodiments, includes before removing a portion of the base material, forming a precursor of the implantable device and after removing a portion of the base material, moving the formed precursor from a first position having a first dimension to a second position having a second dimension that is smaller than the first dimension and heat treating the implantable device.

The method, in some embodiments, includes moving the implantable device from a first position to a second position and heat treating the implantable device. In further embodiments, moving the implantable device from a first position to a second position includes compressing the implantable device.

In some embodiments, tissue engaging portions and a plurality of support members are formed and a first end of the extruded base material is joined to a second end of the extruded base material. In the first state, an inner edge of the inner surface of a first support member and an inner edge of the inner surface of a second support member, in further embodiments, are separated by a first edge dimension. In still further embodiments, in the second state, the inner edge of the inner surface of the first support member and the inner edge of the inner surface of the second support member are separated by a second edge dimension and the second edge dimension is substantially smaller than the first edge dimension.

An embodiment of an implantable device is described. The implantable device includes a planar annular body movable from a first state towards a second state. The annular body includes a plurality of support members. The support members define a cross-section that includes an outer surface having an outer surface dimension and an inner surface having an inner surface dimension. The inner surface dimension is substantially smaller than the outer surface dimension.

In some embodiments, the shape of the cross-section of the support members is selected from the following: an isosceles trapezoid, a semipolygon, a triangle, or a semiellipse and the outer surface defines a base of the cross-section. The implantable device, in some embodiments is a closure element for engaging tissue.

The annular body, in some embodiments, is movable from a pre-deployed configuration towards a deployed configuration and the annular body includes a plurality of tissue engaging portions extending from the annular body. At least two of the tissue engaging portions are separated by a first distance in a deployed configuration and a second distance in a pre-deployed configuration. The first distance in the deployed configuration is smaller than the second distance in the pre-deployed configuration.

In some embodiments, the annular body defines a plane. The annular body is disposed about a central axis extending substantially normal to the plane in the deployed configuration. The annular body is disposed out of the plane in the pre-deployed configuration. The tissue engaging portions are oriented generally towards the central axis in the deployed configuration and generally parallel to the central axis in the pre-deployed configuration.

The annular body, in some embodiments, is biased towards the deployed configuration for biasing at least one of the tissue engaging portions towards another tissue engaging portion. In the first state, an inner edge of the inner surface of a first support member and an inner edge of the inner surface of a second support member, in further embodiments, are separated by a first edge dimension. In the second state, the inner edge of the inner surface of the first support member and the inner edge of the inner surface of the second support member are separated by a second edge dimension. The second edge dimension is substantially smaller than the first edge dimension.

It is to be understood that both the foregoing general description and the following detailed description are exemplary and are intended to provide further explanation of the invention claimed.

The accompanying Figures, which are incorporated in and constitute part of this specification, are included to illustrate and provide a further understanding of the method and system of the invention. Together with the description, the Figures serve to explain the principles of the invention.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings.

FIG. 1A is a top view of a further embodiment of an implantable device in comparison with a conventional implantable device, in accordance with the present invention.

FIG. 1B is a top cross-sectional view of the embodiment an implantable device shown in FIG. 1A.

FIG. 1C is a top cross-sectional view of a conventional implantable device shown in FIG. 1B.

FIG. 1D is a perspective view of an embodiment of an implantable device.

FIG. 1E is a perspective view of another embodiment of an implantable device.

FIG. 2 illustrates an embodiment of a method of fabricating an implantable device according to the present invention.

FIG. 3 illustrates another embodiment of a method of fabricating an implantable device according to the present invention.

FIG. 4 illustrates a further embodiment of a method of fabricating an implantable device according to the present invention.

FIGS. 5A-5G show another embodiment of an implantable device according to the present invention.

FIG. 6 illustrates an embodiment of a method of fabricating an implantable device according to the present invention.

FIG. 7 illustrates a further embodiment of a method of fabricating an implantable device according to the present invention.

FIG. 8 illustrates a still further embodiment of a method of fabricating an implantable device according to the present invention.

FIGS. 9-15 illustrate alternative embodiments of implantable devices in a compressed pre-deployed state in accordance with the present invention in comparison with other implantable devices.

It should be noted that the figures are not drawn to scale and that elements of similar structures or functions are generally represented by like reference numerals for illustrative purposes throughout the figures. It also should be noted that the figures are only intended to facilitate the description of embodiments of the present invention.

### DETAILED DESCRIPTION

The embodiments described herein extend to methods, systems, and apparatus for managing access through tissue. Some of the apparatuses of the present invention are configured to deliver a device for managing access through tissue into an opening formed in and/or adjacent to tissue.

Medical devices may be used in a variety of spaces. It may be desirable to generally reduce the size of medical devices. For example, stents may be inserted into smaller and smaller vasculature, thus making it generally desirable to reduce the pre-deployment size of a stent. In another example, a closure device may be used to close tissue in, for example, a body lumen. In order to reach the desired body lumen, typically a delivery device may be used to reach an access point in the body lumen. To minimize the effects of a procedure on a patient, the reduction in size of the access point may be desirable.

For example, when a stent is deployed, it is initially in a collapsed or smaller state. Once the stent is properly positioned, it can then be expanded to a larger or expanded state. Embodiments of the invention relate to systems and methods that can reduce the size of the collapsed or smaller state of the stent or other medical device. By reducing the size of the collapsed or smaller state, the size of the access point into the body lumen can be similarly reduced. This can advantageously reduce the effects of a procedure on a patient.

These results, whether individually or collectively, can be achieved, according to one embodiment of the present invention, by employing methods, systems, and/or apparatus as shown in the figures and described in detail below.

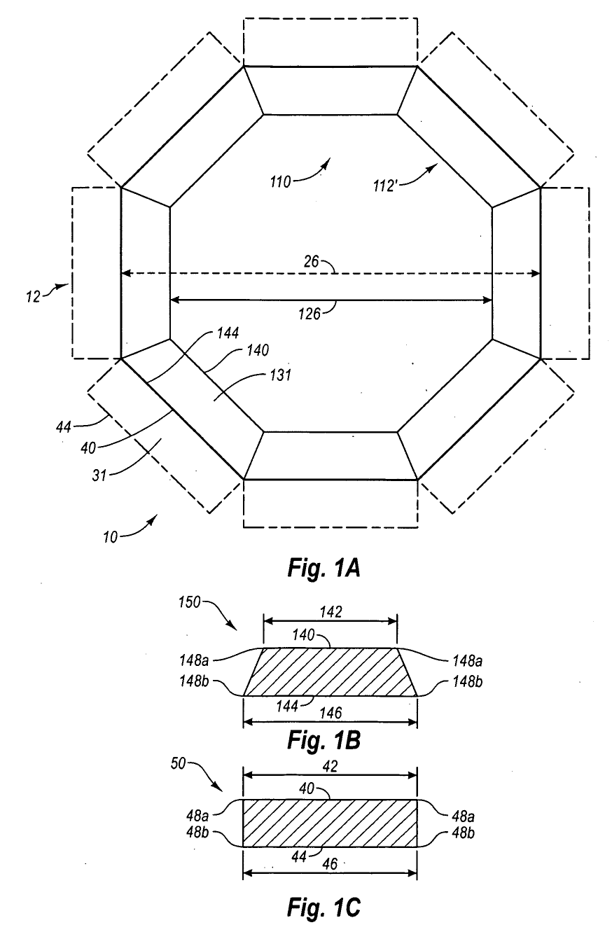

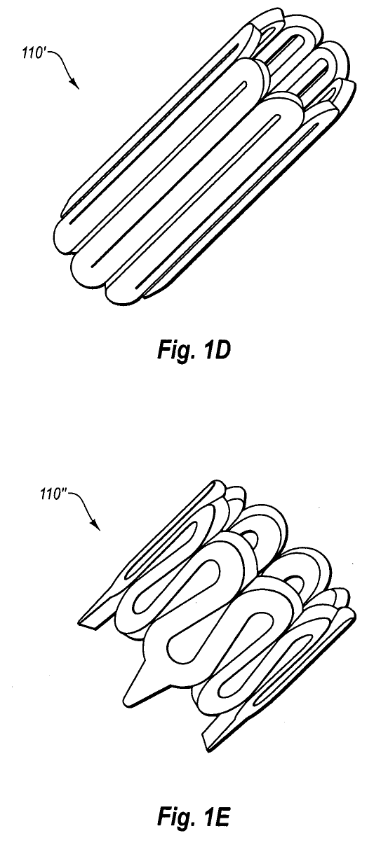

Turning now to the drawings, FIGS. 1A-1E illustrate various embodiments of implantable devices. The devices 10, 110, 110′, 110″ can each transition from a collapsed or smaller state (illustrated in FIG. 1A) to a larger or expanded state. FIGS. 1A-1C illustrate that the implantable device 110 has a collapsed state that is smaller than available in conventional devices without sacrificing the dimensions of the expanded or larger state.

FIG. 1A illustrates a top view of an embodiment of an implantable device 110. FIG. 1A also illustrates another implantable device 10 in phantom. The implantable devices 10, 110 may include a generally annular body 12, 112. The annular body 12, 112 may include support members 31, 131. The annular body 12, 112 may be moveable from a larger state (not shown) toward a smaller state. The smaller state is shown in FIG. 1. The support members 31, 131 may substantially contact one another in the smaller state. However, as shown, the implantable device 110 may have a smaller dimension in the smaller state than the implantable device 10. For example, portions of the implantable devices 10, 110 may be separated by a distance 26, 126, for example an inner diameter, in the smaller state. The distance 26 of the implantable device 10 may be larger than the distance 126 of the implantable device 110. In another example, the dimension may be an inner and/or outer circumference, perimeter, area, width, length, height, diagonal length, radius, volume, and/or other dimension.

FIG. 1B illustrates a portion of the annular body 112 of the embodiment of an implantable device 110 having a reduced dimension in a smaller state shown in FIG. 1A. FIG. 1C illustrates a portion of the annular body 12 of the embodiment of an implantable device 10 shown in phantom in FIG. 1A. The implantable devices 10, 110 may have an inner surface 40, 140 having an inner surface dimension 42, 142 and an outer surface 44, 144 having an outer surface dimension 46, 146. The inner surface dimension 142 of the implantable device 110 may be smaller than the outer surface dimension 146 of the implantable device 110 and the inner surface dimension 42 and outer surface dimension 46 of the implantable device 10.

The implantable device 110 may represent an implantable device that has been processed according to an embodiment of a method of the present invention. The implantable device 10 may represent an implantable device prior to processing according to an embodiment of a method of the present invention.

In the present embodiment, the inner surface dimension 142 of the implantable device 110 is the distance between two edges 148a of the inner surface 140 of the implantable device 110. The inner surface dimension 42 of the implantable device 10 is the distance between two edges 48a of the inner surface 40 of the implantable device 10. The outer surface dimension 146 of the implantable device 110 is the distance between two edges 148b of the outer surface 144 of the implantable device 110. The outer surface dimension 46 of the implantable device 10 is the distance between two edges 48b of the outer surface 44 of the implantable device 10. In other embodiments, the inner and outer surface dimensions 42, 46, 142, 146 of the implantable devices 110, 10 may include other dimensions.

The implantable devices 10, 110 may include a cross-section 50, 150. In the present embodiment, the implantable device 110 has an isosceles trapezoid cross-section. In other embodiments, the implantable device 110 may include other cross-sections 150, such as a semipolygon, a triangle, a semiellipse and/or other cross-sections. The implantable device 10 is shown with a rectangular cross-section 50. Advantageously, an implantable device 110 having annular bodies with a cross section 150 can collapse further that the implantable device 10, which has a rectangular cross-section 50, thereby reducing the size of the collapsed state of the device 10 compared to the collapsed state of the device 10.

FIG. 1D illustrates a perspective view of an embodiment of an implantable device 110′. The implantable device 110′ shown in FIG. 1D may be a stent. The implantable device 110′ may represent an implantable device that has been processed according to an embodiment of a method of the present invention. FIG. 1E illustrates a perspective view of an embodiment of an implantable device 110″. The implantable device 110″ shown in FIG. 1E may be a closure element, such as a clip. The implantable device 110″ may represent an implantable device that has been processed according to an embodiment of a method of the present invention.

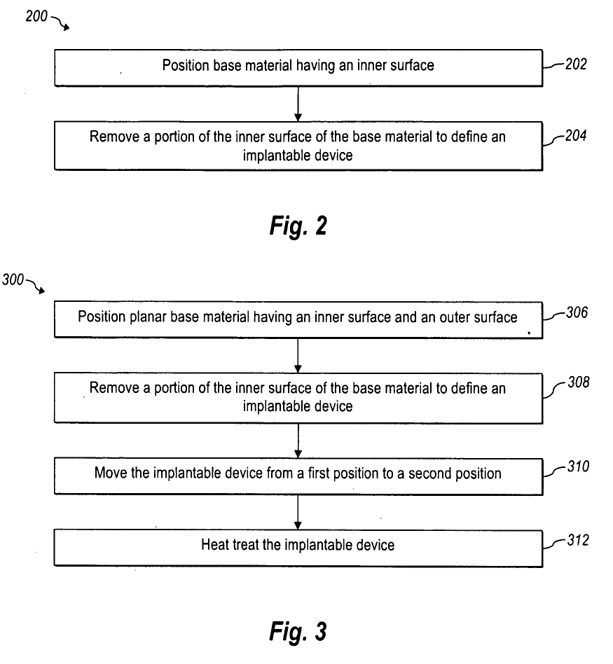

FIG. 2 illustrates an embodiment of a method 200 of fabricating an implantable device. The present embodiment is described with reference to the implantable devices 10, 110, 110′, 110″ described in connection with FIGS. 1A-1E. The method 200 may include positioning a base material, as represented by block 202. In the present embodiment, the base material may include an inner surface. Positioning a base material may include placing a base material in a fixture, such as a jig, a vise, another fixture, or combinations thereof.

A portion of the inner surface of the base material may be removed, as represented by block 204. The portion of the inner surface of the base material may be removed using various techniques. For example, the portion of the inner surface of the base material may be removed using laser cutting, photolithography, chemical etching, EDM, milling, hydro-cutting, other removal processes, or combinations thereof.

For example, the base material may be removed by laser cutting. To achieve the desired cross sectional shape, the laser, in one embodiment, is angled. When using photolithography, the size of the mask on the inner surface 142 may be thinner than the mask on the surface 144. During photolithography, the desired cross-sectional shape, such as trapezoidal, can be obtained.

Removing a portion of the inner surface of the base material may define an implantable device. An implantable device may include a closure element, such as those described in U.S. patent application Ser. No. 11/767,818, entitled “Methods, Devices, and Apparatus for Managing Access Through Tissue”, and filed Jun. 25, 2007, which is hereby incorporated by reference in its entirety, a stent, or other implantable devices.

FIG. 3 illustrates another embodiment of a method 300 of fabricating an implantable device. The method 300 may include positioning a base material having an inner surface and an outer surface, as represented by block 306. Positioning a base material may include placing a base material in a fixture, as described herein.

A portion of the inner surface of the base material may be removed, as represented by block 308. Removing a portion of the inner surface of the base material may define an implantable device, as described herein. Removing a portion of the inner surface typically results in a change to the shape of the base material and often changes at least one dimension, such as a diameter of a collapsed state of an implantable device or of a device for deploying an implantable device.

The implantable device may be moved from a first position to a second position, as represented by block 310. Moving the implantable device from a first position to a second position may include expanding, compressing, rotating, flexing, other movements of the implantable device, or combinations thereof.

The implantable device may be heat treated, as represented by block 312. In the present embodiment, the implantable device may be heat treated while it is moved from the first position to the second position. The implantable device may be fixed in the second position while it is being heat treated. Heat treating the implantable device may include heating the implantable device to a temperature above its austenitic finish temperature, followed by water quenching.

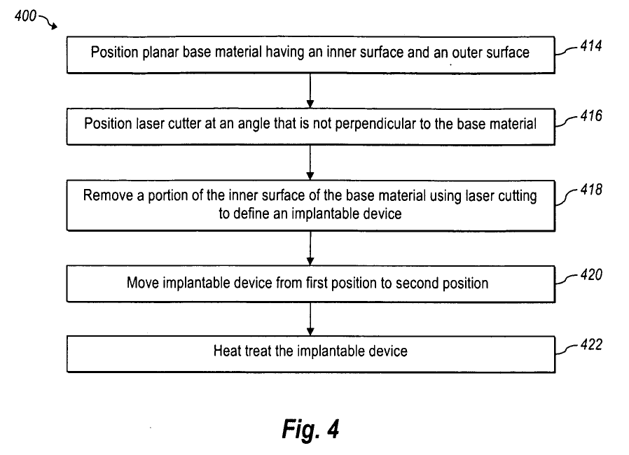

FIG. 4 illustrates a further embodiment of a method 400 of fabricating an implantable device. The method 400 may include positioning a base material having an inner surface and an outer surface, as represented by block 414. Positioning a base material may include placing a base material in a fixture, as described herein.

In the present embodiment, a laser cutter may be positioned at an angle that is not perpendicular to the base material, as represented by block 416. Positioning a laser cutter at an angle that is not perpendicular to the base material may include positioning the base material and/or the laser cutter relative to each other such that the laser may cut the base material in a non-perpendicular fashion.

A portion of the inner surface of the base material may be removed, as represented by block 418. In the present embodiment, the portion of the inner surface of the base material may be removed using laser cutting. Removing a portion of the inner surface of the base material may define an implantable device, as described herein. Additional portions of the inner surface of the base material may be removed or all portions to be removed may be removed at the same time.

The implantable device may be moved from a first position to a second position, as represented by block 420. Moving the implantable device from a first position to a second position may include expanding, compressing, rotating, flexing, other movements of the implantable device, or combinations thereof, as described herein.

The implantable device may be heat treated, as represented by block 422. In the present embodiment, the implantable device may be heat treated while it is moved from the first position to the second position, as described herein.

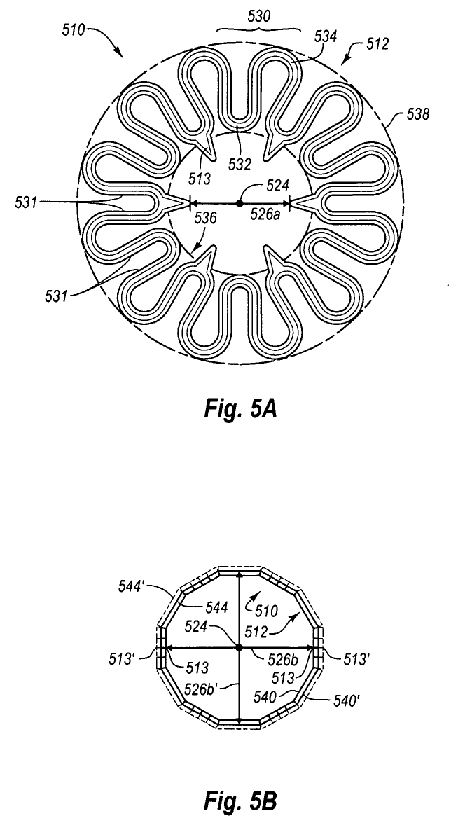

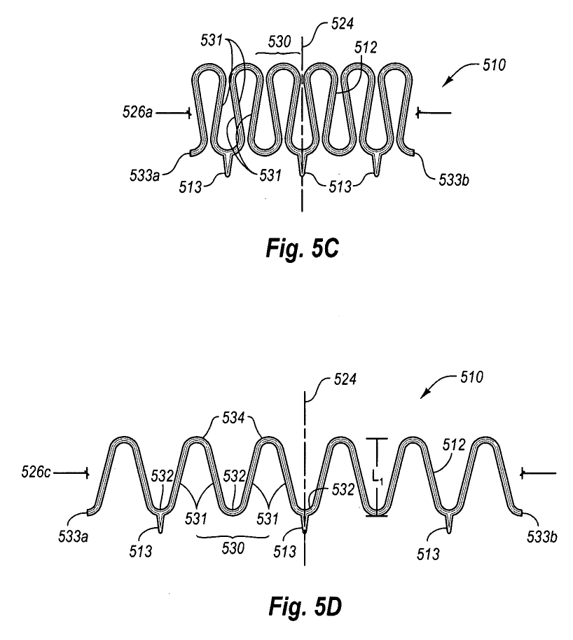

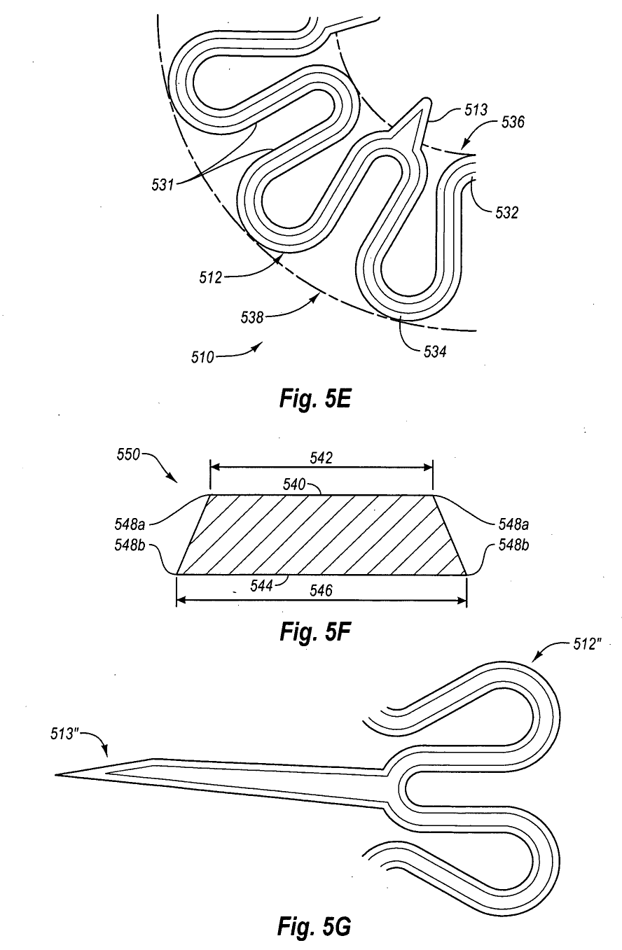

FIGS. 5A-5G illustrate another embodiment of an implantable device 510. The implantable device 510, in the present embodiment, may be a closure device. The implantable device 510 may be used for closing an incision, puncture, or other passage through tissue or access point. In some embodiments, the implantable device 510 may close communication with a blood vessel or other body lumen. The implantable device 510 may include a body 512. The body 512 may include support members 531. In the present embodiment, the body 512 may be generally annular in shape and/or may surround a central axis 524. As used herein, an “annular-shaped body” may include any hollow body, e.g., including one or more structures surrounding an opening, whether the body is substantially flat or has a significant thickness or depth. Thus, although an annular-shaped body may be circular, it may include other noncircular shapes as well, such as elliptical or other shapes that are asymmetrical about a central axis. In other embodiments, the body 512 may include other shapes and/or may not have a central axis 524.

The implantable device 510 may include a plurality of tissue engaging portions 513 extending from the body 512. The tissue engaging portions 513 may include edges (not shown) and/or tip portions (not shown). Portions of the tissue engaging portions 513 may include edges and/or tip portions that are sharp and/or obtuse. In some embodiments, the tissue engaging portions 513 may not have edges such that they are generally rounded.

In the present embodiment, the body 512 may include a plurality of looped or curved elements 530 that may be connected to one another to form the body 512. Each looped element 530 may include an inner or first curved region 532 and an outer or second curved region 534. The first and second curved regions 532, 534 may be out of phase with one another and/or may be connected alternately to one another, thereby defining an endless sinusoidal pattern. Alternatively, other generally zigzag patterns may be provided that repeat periodically, e.g., saw tooth or square tooth patterns (not shown), instead of a sinusoidal pattern, thereby defining inner and outer regions that may alternate about the body 512.

FIG. 5A shows the implantable device 510 in a deployed configuration. In the present embodiment, when the implantable device 510 is in the deployed configuration, the first curved regions 532 may define an inner periphery 536 of the body 512 and the implantable device 510, and the second curved regions 534 may define an outer periphery 538. The deployed configuration, in the present embodiment, may be a substantially planar configuration. In other embodiments, the deployed configuration may be another type of configuration.

The plurality of tissue engaging portions 513 may be biased to extend towards one another. In the present embodiment, the tissue engaging portions 513 may be biased generally inwardly into the space bounded by the inner periphery 536. In further embodiments, the tissue engaging portions 513 may be biased toward the central axis 524. In further embodiments, at least two of the tissue engaging portions 513 may be biased to extend towards each other.

In the present embodiment, the tissue engaging portions 513 may be disposed on the first curved regions 532 and/or oriented toward the central axis 524 when the implantable device 510 is in the deployed configuration. The tissue engaging portions 513 may be provided in pairs opposite from one another, as in the present embodiment. The tissue engaging portions 513 may be provided symmetrically with respect to the central axis 524 or may not be provided symmetrically.

Additionally, as shown in FIGS. 5A, 5C-5E, and 5G, the tissue engaging portions 513 may be disposed on alternating first curved regions 532. Thus, at least one period of a zigzag pattern may be disposed between adjacent tissue engaging portions 513, which may enhance flexibility of the implantable device 510.

In the deployed configuration, shown in FIG. 5A, the tissue engaging portions 513 may be separated by a first distance 526a. In a pre-deployed configuration, shown in FIGS. 5B-5C, the tissue engaging portions 513 may be separated by a second distance 526b. In the present embodiment, the first and second distances 526a, 526b may be measured from the tip portion (not shown) of two tissue engaging portions 513. In other embodiments, the first and second distances 526a, 526b may be measured from another portion of the tissue engaging portions 513, for example from the base (not shown) of the tissue engaging portions 513. The first distance 526a, in the present embodiment, may be smaller than the second distance 526b, such that the distance 526a in the deployed configuration may be smaller than the distance 526b in the pre-deployed configuration.

The distances 526a, 526b may vary before deployment, pre-deployment, and/or when providing access through the tissue post deployment. In the present embodiment, before being deployed in tissue, the implantable device 510 may be substantially in the pre-deployed configuration such that two tissue engaging portions 513 may be separated by about the second distance 526b. When deployed in tissue, the implantable device 510 may be substantially in the deployed configuration such that the two tissue engaging portions 513 may be separated by about the first distance 526a. When providing access to the tissue after being deployed in tissue, the implantable device 510 may be moved from substantially the deployed configuration substantially toward and/or to the pre-deployed configuration.

As shown in FIG. 5B, the body 512 and/or the tissue engaging portions 513 may be deflected into the pre-deployed configuration. In the present embodiment, the tissue engaging portions 513 may extend transversely with respect to a plane defined in the deployed configuration, thereby defining the pre-deployed configuration for the implantable device 510. In other embodiments, the body 512 and/or the tissue engaging portions 513 in the pre-deployed configuration may not extend transversely with respect to a plane defined in the deployed configuration. For example, the body 512 and/or the tissue engaging portions 513 in the pre-deployed configuration may remain in a plane defined in the deployed configuration. In another example, the body 512 and/or the tissue engaging portions 513 in the pre-deployed configuration may move out of although not completely transverse to a plane defined in the deployed configuration.

In the present embodiment, the tissue engaging portions 513 may be oriented substantially parallel to the central axis 524 in the pre-deployed configuration, as shown in FIG. 5C. In this pre-deployed configuration, the body 512 may have a generally annular shape defining a length (not shown), which may extend generally parallel to the central axis 524, and may correspond generally to an amplitude of the zigzag pattern. The body 512 may be sufficiently flexible such that the implantable device 510 may assume a generally circular or elliptical shape, as shown in FIG. 5B, e.g. substantially conforming to an exterior surface of a delivery device (not shown) used to deliver the implantable device 510.

The tissue engaging portions 513 and/or body 512 may be biased to move from the pre-deployed configuration towards the deployed configuration of FIG. 5A. Thus, with the tissue engaging portions 513 in the pre-deployed configuration, the tissue engaging portions 513 may penetrate and/or be engaged with tissue at a puncture site. When the implantable device 510 is released, the tissue engaging portions 513 may attempt to return towards one another (i.e. the distance may decrease from the second distance 526b toward the first distance 526a) as the implantable device 510 moves towards the deployed configuration, thereby drawing the engaged tissue together and substantially closing and/or sealing the puncture site.

The looped elements 530 may distribute stresses in the implantable device 510 as it is moved between the deployed and pre-deployed configurations, thereby generally minimizing localized stresses that may otherwise plastically deform, break, and/or otherwise damage the implantable device 510 during delivery. In addition, when the implantable device 510 is in the pre-deployed configuration, the looped elements 530 may be movable between a compressed state, such as that shown in FIGS. 5B-5C, and an expanded state, such as that shown in FIG. 5D (where opposite ends 533a, 533b are connected to one another). The body 512 may be biased towards the expanded state, but may be moved (e.g. compressed) to the compressed state, e.g., by constraining the implantable device 510. Alternatively, only a portion of the body 512 may be biased towards the expanded state. For example, in the present embodiment, the first curved regions 532 and/or the looped elements 530 may be biased towards the compressed state. Furthermore, the looped elements 530 may reduce the force exerted on the implantable device 510 to transition the implantable device 510 from the deployed configuration to the pre-deployed configuration before loading onto a delivery device (not shown).

With the implantable device 510 in the pre-deployed configuration, the support members 531 may be moved (circumferentially and/or radially compressed in the present embodiment) to the compressed state until the device 510 defines a first distance 526b (i.e. diameter or circumference), such as that shown in FIGS. 5B-5C. in the present embodiment, the support members 531 may contact each other in the compressed pre-deployed state. The implantable device 510 may be constrained in the compressed state, e.g., by loading the implantable device 510 onto a carrier assembly of a delivery device (not shown), such as U.S. patent application Ser. No. 10/356,214, entitled “Clip Applier and Methods of Use”, filed Jan. 30, 2003. When released from the constraint, e.g., when deployed from the carrier assembly, the implantable device 510 may move (automatically expand in the present embodiment) towards the expanded state, such as that shown in FIG. 5D, thereby defining a third distance 526c, for example a second diameter or circumference. Thus, the looped elements 530 may facilitate reducing the profile of the implantable device 510 during delivery, e.g., to facilitate introducing the implantable device 510 through a smaller puncture or passage. Once the implantable device 510 is deployed entirely from the delivery device, the looped elements 530 may resiliently expand as the implantable device 510 returns towards the deployed configuration.

Referring generally to FIGS. 5B, 5E, and 5F, the implantable device 510 may include an inner surface 540 having an inner surface dimension 542 (shown in FIG. 5F) and an outer surface 544 having an outer surface dimension 546 (shown in FIG. 5F). In the present embodiment, the inner surface dimension 542 of the implantable device 510 is the distance between two edges 548a of the inner surface 540 of the implantable device 510. The outer surface dimension 546 of the implantable device 510, in the present embodiment, is the distance between two edges 548b of the outer surface 544 of the implantable device 510. In other embodiments, the inner and outer surface dimensions 542, 546 of the implantable device 510 may include other dimensions as described herein. In the present embodiment, the inner surface dimension 542 may be smaller than the outer surface dimension 546 of the implantable device 510.

FIG. 5B also illustrates another implantable device 510′ (in phantom) in a pre-deployed configuration. Like the implantable device 510, the implantable device 510′ includes an annular body 512′, tissue engaging portions 513′, an inner surface 540′ having an inner surface dimension (not shown), and an outer surface 544′ having an outer surface dimension (not shown). Furthermore, the implantable device 510′ may have a second distance 526b′ separating two tissue engaging portions 513′. However, the second distance 526b′ of the implantable device 510′ is larger than the second distance 526b the implantable device 510. One of the reasons why the second distance 526b′ of the implantable device 510′ is larger than the second distance 526b of the implantable device 510, may include that the inner surface 540′ of the implantable device 510′ is has the same dimension as the outer surface 544′ of the implantable device 510′ compared to the smaller inner surface dimension 542 versus the outer surface dimension 546 of the implantable device 510.

An implantable device 510 with an inner surface dimension 542 that is smaller than an outer surface dimension 546 may provide the advantage of being smaller (i.e. having a smaller second distance 526b) in a compressed pre-deployed state than an implantable device 510′ with an inner surface dimension and an outer surface dimension that are substantially equal. For example, it may be desirable to have an implantable device 510 with a smaller second distance 526b in a compressed pre-deployed state to reduce the overall size of an apparatus for deploying an implantable device (not shown). This reduction in size may facilitate introducing the implantable device 510 through a smaller puncture or passage.

FIG. 5G illustrates another embodiment of a tissue engaging portion 513″. The tissue engaging portion 513″ of this alternative embodiment may be functionally similar to that of the tissue engaging portions 513 previously described above and shown in FIGS. 5A-5F in most respects, wherein certain features will not be described in relation to the alternative embodiments wherein those components may function in the manner as described above and are hereby incorporated into the alternative embodiment described below.

In the present embodiment the longer tissue engaging portion 513″ may extend from the body 512″. Longer tissue engaging portions 513″ may include edges and/or tip portions. Portions of the longer tissue engaging portions 513″ may include edges and/or tip portions that are sharp and/or obtuse. In some embodiments, the longer tissue engaging portions 513″ may not have edges such that they are generally rounded.

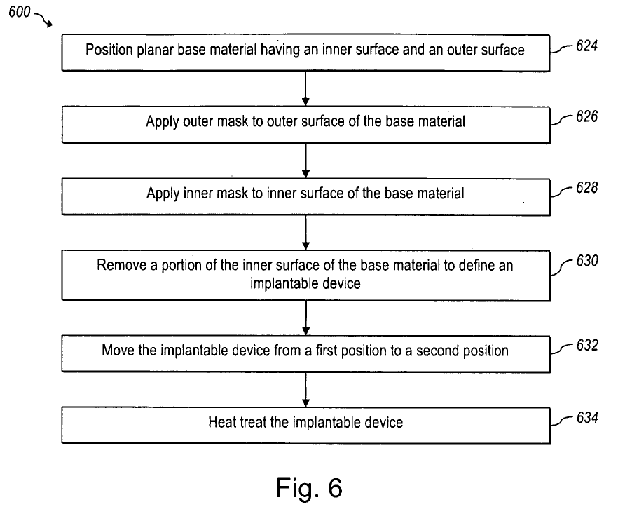

FIG. 6 illustrates an embodiment of a method 600 of fabricating an implantable device. The method 600 may include positioning a base material having an inner surface and an outer surface, as represented by block 624. Positioning a base material may include placing a base material in a fixture, as described herein.

An outer mask may be applied to an outer surface of the base material, as represented by block 626. Applying an outer mask to an outer surface of the base material may include selecting a desired shape of an outer surface of an implantable device. For example, the general shape of the outer surface 544 of the implantable device 510 shown in FIGS. 5A-5G may be selected.

An inner mask may be applied to an inner surface of the base material, as represented by block 628. Applying an inner mask to an inner surface of the base material may include selecting a desired shape of an inner surface of an implantable device, a desired general cross-section of a portion (i.e. the body) of the implantable device, and/or other design considerations. For example, the general shape of the inner surface 540 of the implantable device 510 shown in FIGS. 5A-5G may be selected and particularly the cross-section 550 of the implantable device 510 shown in FIG. 5F.

A portion of the inner surface of the base material may be removed, as represented by block 530. Removing a portion of the inner surface of the base material may define an implantable device. An implantable device may include a closure element, such as the implantable device 510 and/or other implantable devices (i.e. stents, etc.) as described above.

The base material that does not have either an inner or an outer mask may be generally removed. The material between the inner mask and outer mask may not be generally removed. However, in some embodiments, some portions of the base material covered by an inner and/or outer mask may be partially removed. For example, where chemical etching is used, the etching chemical may remove more material than the unmasked areas potentially removing a portion of the base material covered by an inner and/or outer mask. The inner and/or outer mask configurations may be designed to compensate for any overetching that may occur.

In the present embodiment, the inner mask may have a smaller dimension than the outer mask. For example, the overall surface area of the inner mask may be smaller than the overall surface area of the outer mask. In embodiments, where the inner mask has a smaller dimension than the outer mask, the material that is masked by the outer mask but not by the inner mask may generally taper from the outer mask to the inner mask. For example, if an inner mask were placed on the inner surface 540 of the implantable device 510 and an outer mask were placed on the outer surface 544 of the implantable device 510 shown in FIG. 5F, the material may be generally removed to create the cross-section 550 shown in FIG. 5F.

In some embodiments, the implantable device may already be defined in shape, such as implantable device 10. In these embodiments, an inner and/or an outer mask may be applied to create a desired cross-section as described above although the body of the implantable device was previously defined.

The implantable device may be moved from a first position to a second position, as represented by block 632. Moving the implantable device from a first position to a second position may include expanding, compressing, rotating, flexing, other movements of the implantable device, or combinations thereof, as described herein. For example, the implantable device may be moved from a pre-deployed configuration to the deployed configuration. In another example, the implantable device may be formed in an expanded oversize configuration to provide space for removing material from a sheet of material such that the implantable device may be moved from the expanded oversize configuration to the deployed configuration.

The implantable device may be heat treated, as represented by block 634. In the present embodiment, the implantable device may be heat treated while it is moved from the first position to the second position, as described herein.

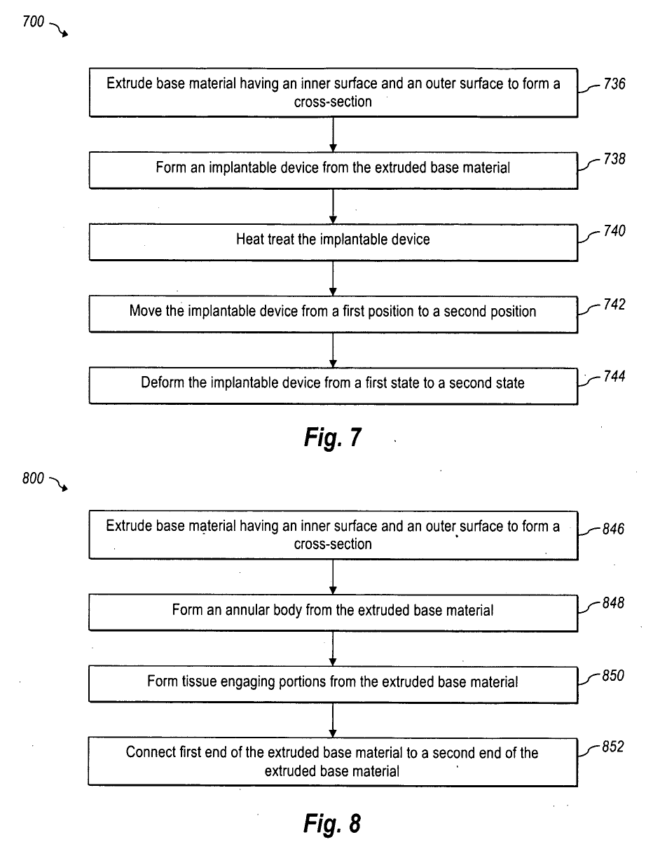

FIG. 7 illustrates a further embodiment of a method 700 of fabricating an implantable device. The method 700 may include extruding a base material, as represented by block 736. The extruded base material may include an inner surface and an outer surface that may form a cross-section.

An implantable device may be formed from the extruded base material, as represented by block 738. Forming an implantable device from the extruded base material may include connecting two ends of the extruded base material, e.g., by welding, adhesive bonding, other connection processes, or combinations thereof.

The implantable device may be moved from a first position to a second position, as represented by block 740. Moving the implantable device from a first position to a second position may include expanding, compressing, rotating, flexing, other movements of the implantable device, or combinations thereof, as described herein.

The implantable device may be heat treated, as represented by block 742. In the present embodiment, the implantable device may be heat treated while it is moved from the first position to the second position, as described herein.

The implantable device may be deformed from a first state to a second state, as represented by block 744. For example, the implantable device may be deformed from a deployed state to a pre-deployed state, from an expanded pre-deployed state to a compressed pre-deployed state, from a compressed pre-deployed state to a deployed state, other deformations from a first to a second state, or combinations thereof.

FIG. 8 illustrates a still further embodiment of a method 800 of fabricating an implantable device. The method 800 may include extruding a base material, as represented by block 846. The extruded base material may include an inner surface and an outer surface that may form a cross-section, as described herein.

An implantable device may be formed from the extruded base material. Forming an implantable device from the extruded base material may include forming an annular body, as represented by block 848, and/or forming tissue engaging portions, as represented by block 850.

Forming an annular body may include winding the extruded base material into an enclosed loop. For example, in embodiments where the implantable device is a closure element, such as implantable device 510 shown in FIGS. 5A-5G, the extruded base material may be wound to form a plurality of looped or curved elements 530.

Forming tissue engaging portions may include bending the extruded base material to form tissue engaging portions using conventional methods. Alternatively or in addition, tissue engaging portions may be formed separately and attached to the body, for example, by welding.

A first end of the extruded base material may be connected to a second end of the extruded base material, as represented by block 852. Connecting a first and second end of the extruded base material may include joining the two ends by, for example, welding, other connection processes, or combinations thereof.

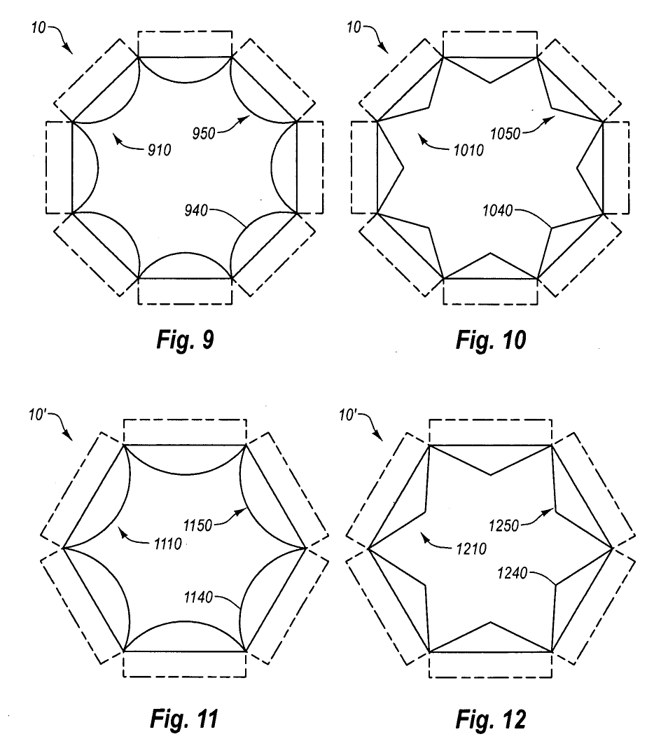

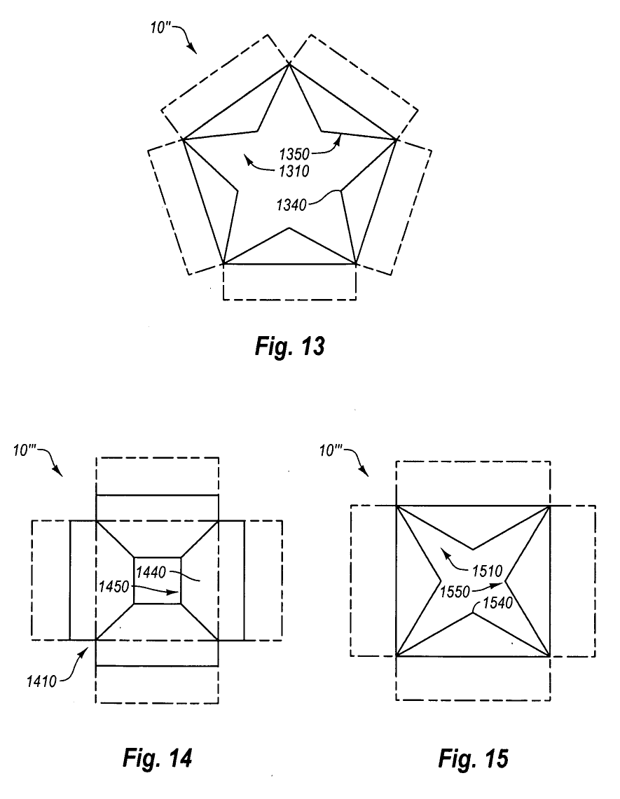

FIGS. 9-15 illustrate alternative embodiments of implantable devices 910, 1010, 1110, 1210, 1310, 1410, 1510 in a compressed pre-deployed state in accordance with the present invention in comparison with other implantable devices 10, 10′, 10″, 10′″. FIGS. 9 and 11 illustrate implantable devices 910, 1110 with generally semi-circular cross-sections 950, 1150. The implantable devices 910, 1110 may include curved inner surfaces 940, 1140.

FIGS. 10, 12, 13, and 15 illustrate implantable devices 1010, 1210, 1310, 1510 with generally triangular cross-sections 1050, 1250, 1350, 1550. The implantable devices 1010, 1210, 1310, 1510 may include curved inner surfaces 1040, 1240, 1340, 1540.

FIG. 14 illustrates an implantable device 1410 with a generally semi-polygonal cross-section 1450. In comparison with the isosceles trapezoid cross-section 150 shown in FIG. 1D, the generally semi-polygonal cross-section 1450 may not remove material from the inner surface (not shown) to the outer surface (not shown), but rather may only remove a portion of the material between the inner and the outer surfaces.

The inner surfaces 1040, 1140, 1240, 1340, 1440, 1540 may provide the advantage of being smaller in a compressed pre-deployed state than the implantable devices 10, 10′, 10″, 10′″ with an inner surface dimension (not shown) and an outer surface dimension (not shown) that are substantially equal.

The invention is susceptible to various modifications and alternative means, and specific examples thereof have been shown by way of example in the drawings and are herein described in detail. It should be understood, however, that the invention is not to be limited to the particular devices or methods disclosed, but to the contrary, the invention is to cover all modifications, equivalents, and alternatives falling within the spirit and scope of the claims.

What is claimed is:

1. A method of fabricating an implantable device, the method comprising:
positioning a planar base material having a first inner surface having a first inner surface dimension and a first outer surface having a first outer surface dimension;
removing a portion of the first inner surface of the base material to define an annular body movable from a first state towards a second state, the annular body including a second inner surface having a second inner surface dimension and a second outer surface having a second outer surface dimension, wherein the second inner surface dimension is smaller than the first inner surface dimension, the first outer surface dimension, and the second outer surface dimension.

2. The method of claim 1, wherein second inner surface and the second outer surface define a cross-section the shape of which is selected from the following: an isosceles trapezoid, a semipolygon, a triangle, or a semiellipse and wherein the second outer surface defines a base of the cross-section.

3. The method of claim 2, wherein removing a portion of the base material further comprises removing one or more portions from the base material using laser cutting.

4. The method of claim 3, wherein removing a portion of the base material further comprises using a laser at an angle that is not perpendicular to the first outer surface or second outer surface.

5. The method of claim 2, wherein removing a portion of the base material further comprises removing a portion from the base material using photochemical etching.

6. The method of claim 5, wherein removing a portion of the base material further comprises selectively adding an outer mask having an outer mask dimension to the first outer surface of the base material and selectively adding an inner mask having an inner mask dimension to the first inner surface of the base material to form the cross-section, wherein the outer mask dimension is larger than the inner mask dimension.

7. The method of claim 1, wherein the first inner surface dimension and the first outer surface dimension are substantially the same dimension, the method further comprising removing a portion of the first outer surface of the base material and wherein the first outer surface dimension is substantially larger than the second outer surface dimension.

8. The method of claim 7, wherein the annular body further comprises a plurality of adjacent support members, the method further comprising:
before removing a portion of the base material, forming a precursor of the implantable device; and
after removing a portion of the base material, moving the formed precursor from a first position having a first dimension to a second position having a second dimension that is smaller than the first dimension and heat treating the implantable device.

9. The method of claim 1, the method further comprising:
moving the implantable device from a first position to a second position; and
heat treating the implantable device.

10. The method of claim 9, wherein moving the implantable device from a first position to a second position further comprises compressing the implantable device.

11. An implantable device, comprising:
a planar annular body movable from a first state towards a second state, the annular body comprising a plurality of support members, the support members defining a cross-section including an outer surface having an outer surface dimension and an inner surface having an inner surface dimension, wherein the inner surface dimension is substantially smaller than the outer surface dimension.

12. The implantable device of claim 11, wherein the shape of the cross-section of the support members is selected from the following: an isosceles trapezoid, a semipolygon, a triangle, or a semiellipse and wherein the outer surface defines a base of the cross-section.

13. The implantable device of claim 11, further comprising a closure element for engaging tissue.

14. The implantable device of claim 13, wherein the annular body is movable from a pre-deployed configuration towards a deployed configuration and wherein the annular body further comprises a plurality of tissue engaging portions extending from the annular body, at least two of the tissue engaging portions being separated by a first distance in a deployed configuration and a second distance in a pre-deployed configuration, wherein the first distance in the deployed configuration is smaller than the second distance in the pre-deployed configuration.

15. The implantable device of claim 14, the annular body further defining a plane, the annular body being disposed about a central axis extending substantially normal to the plane in the deployed configuration, the annular body being disposed out of the plane in the pre-deployed configuration, the tissue engaging portions being oriented generally towards the central axis in the deployed configuration, and generally parallel to the central axis in the pre-deployed configuration.

16. The device of claim 15, wherein the annular body is biased towards the deployed configuration for biasing at least one of the tissue engaging portions towards another tissue engaging portion.

17. The implantable device of claim 11, wherein in the first state an inner edge of the inner surface of a first support member and an inner edge of the inner surface of a second support member are separated by a first edge dimension, wherein in the second state the inner edge of the inner surface of the first support member and the inner edge of the inner surface of the second support member are separated by a second edge dimension, and wherein the second edge dimension is substantially smaller than the first edge dimension.

18. A method of fabricating an implantable device, the method comprising:
extruding a base material having an inner surface and an outer surface to form a cross-section selected from the following: an isosceles trapezoid, a semipolygon, a triangle, or a semiellipse and wherein the outer surface defines a base of the cross-section;
forming an implantable device from the extruded base material, the implantable device comprising an annular body movable from a first state towards a second state; and
deforming the implantable device from the first state where the implantable device has a first dimension to the second state where the implantable device has a second dimension, wherein the first dimension in the first state is substantially larger than the second dimension in the second state.

19. The method of claim 18, further comprising forming tissue engaging portions and a plurality of support members and joining a first end of the extruded base material to a second end of the extruded base material.

20. The method of claim 19, wherein in the first state an inner edge of the inner surface of a first support member and an inner edge of the inner surface of a second support member are separated by a first edge dimension, wherein in the second state the inner edge of the inner surface of the first support member and the inner edge of the inner surface of the second support member are separated by a second edge dimension, and wherein the second edge dimension is substantially smaller than the first edge dimension.
